(19) United States
(12) Patent Application Publication
(10) Pub. No.: US 2026/0219271 A1
(43) Pub. Date: Jul. 30, 2026

(54) IN VITRO DIAGNOSTIC METHOD FOR DETECTING THE PRESENCE OF A TARGET BY USING STABILIZED MEMBRANE PROTEINS

(71) Applicant: Cube Biotech GmbH, Monheim (DE)

(72) Inventors: Barbara Maertens, Köln (DE); Jan Kubicek, Köln (DE); Roland Fabis, Leverkusen (DE); Philipp Timo Hanisch, Langenfeld (DE); Sergej Balanda, Düsseldorf (DE)

(21) Appl. No.: 19/111,256

(22) PCT Filed: Jul. 12, 2023
(86) PCT No.: PCT/EP2023/074569

§ 371 Date: Mar. 11, 2026

Related U.S. Application Data
(60) Provisional application No. 63/406,888, filed on Sep. 15, 2022.

(51) Int. Cl. G01N 33/569; G01N 33/543; G01N 33/58

(52) U.S. Cl. CPC G01N 33/56983; G01N 33/54387; G01N 33/587; G01N 2333/165; G01N 2469/10; G01N 2800/26

(57) ABSTRACT

The technology pertains to in vitro diagnostic methods, kits, membrane proteins and test devices for detecting the presence and/or absence of a target in a biological sample, wherein said target binds to at least one epitope of an isolated membrane protein, or at least to a fragment of said isolated membrane protein comprising at least one epitope of said isolated membrane protein binding to said target.

### DESCRIPTION OF THE INVENTION

The present invention relates to novel methods and test devices for the use of isolated stabilized membrane proteins for the detection of biological targets like host cell's viral receptors as detector molecules for the detection of a virus infection.

### BACKGROUND OF THE INVENTION

Analysis of a biological sample to discern its characteristics, and to get information about various biological targets is in demand both in biology and medicine. A variety of methods may be employed for analyzing the biological sample to detect, inter alia, the presence, absence, concentration, and/or spatial distribution of the biological targets. For example, detection of proteins in histological sections or cytological preparations may be performed using histochemistry, immunohistochemistry (IHC), or immunofluorescence. However, many of the existing techniques for detecting targets in a biological sample have limitations in terms of sensitivity, accuracy and/or multiplexing abilities.

For example, clinical detection of viruses is usually accomplished using any one of a variety of methods. For example, virus particles or nucleic acids may be isolated from a biological sample (e.g., nasopharyngeal aspirates, throat swabs, blood fluids, fecal material, etc.). A retrospective diagnosis may be made by serology. Complement Fixation Tests (CFT) are most widely used in this method, although hemagglutination inhibition (HAI) and enzyme immunoassays (EIA) may be used to give a type-specific diagnosis. For more rapid diagnosis, either antigen detection or RNA detection may be performed. Antigen detection may be done by IFT or EIA, however, to achieve the highest level of sensitivity and specificity, RNA detection by reverse transcriptase polymerase chain reaction (RT-PCR) is used. However, the latter is expensive and technically demanding.

For example, around ⅔ of HBV infections are asymptomatic without a clear clinical manifestation of hepatitis. Therefore, laboratory diagnostics are essential for the reliable detection of HBV infection. In the early phase of HBV infection, HBV DNA (detection by PCR) is the first positive marker of infection (but usually only several weeks after exposure). A few weeks later, viral antigen follows as the first serological marker (HBsAg, HBcAg, HBeAg, detection by ELISA). With the onset of acute hepatitis B symptoms, virus-specific antibodies become detectable in immunocompetent individuals (anti-HBs, anti-HBc, anti-HBe ELISA). While antibodies can often only be detected sometime after the onset of the disease, PCR offers a high degree of diagnostic certainty.

The disadvantage of state-of-the-art testing methods are: (i) variable components of the viruses are detected (viral proteins, viral genome). (ii) PCR is also time and material consuming. (iii) Most detection is done in the laboratory and requires at least one working day, i.e., test results are available the next day at the earliest. (iv) Rapid tests often have insufficient sensitivity and accuracy.

Therefore, the object of the present disclosure is to provide diagnostic in vitro methods for the detection of a pathogen virus, wherein the method shows high specificity, sensitivity and can be performed without a laboratory in a short time.

### SUMMARY OF THE DISCLOSURE

The present disclosure pertains to novel methods and test devices for the use of isolated stabilized membrane proteins for the detection of biological targets like host cell's viral receptors as detector molecules for the detection of a virus infection. For example, the binding of a virus to the receptor enables the virus to infect and thus represents a highly conserved and unchanging process. A rapid test based on this method is characterized by high specificity, sensitivity and rapid performance without the need for a laboratory.

The methods for detecting a target in a biological sample according to the present disclosure generally comprise (a) contacting said biological sample with an isolated membrane protein and with a polymer and/or copolymer that can solubilize and stabilize said membrane protein; and (b) detecting the formation of the target-protein complex comprising said target and said membrane protein, or said fragment thereof, in the biological sample.

In particular, the method of the present disclosure is using a host cell's viral receptor as a detector. The binding of the virus to the receptor enables the virus to infect and thus represents a highly conserved and unchanging process. A rapid test based on this method is characterized by high specificity, sensitivity and rapid performance without the need for a laboratory.

Often, the host cell receptors are water-insoluble membrane proteins. To isolate them from the biomembrane and bring them into solution, state-of-the-art solubilizing detergents are used (e.g. SDS, non-ionic glucosides or maltosides, Triton X-100, CHAPS). With these detergents, the lipid bilayer surrounding the membrane protein is destroyed. However, without the surrounding lipid bilayer of the biomembrane, unfolding of the protein often occurs, especially in human membrane proteins, resulting in irreversible destruction of function. The specificity for binding the virus is also lost in the process. For example, with a non-aromatic, detergent-free polymer (DIBMA=diisobutyl-ene/maleic acid copolymer) which, in combination with phospholipids, maintains the native lipid bilayer in which the membrane protein is normally incorporated. With DIBMA, in the prior art it was already demonstrated that membrane proteins can be functionally and detergent-free solubilized and stabilized by wrapping them with a polymer in a so-called nanodisc. Therefore, with DIBMA or DIBMA variants, it is possible to stabilize host cell viral receptors in nanodiscs in such a way that the specificity for binding the virus is maintained and use as.

Therefore, in a first aspect the present disclosure pertains to in vitro diagnostic methods for detecting the presence and/or absence of a target in a biological sample, wherein said target binds to at least one epitope of an isolated membrane protein, or at least to a fragment of said isolated membrane protein comprising at least one epitope of said isolated membrane protein binding to said target, wherein the method comprises:
(a) contacting said biological sample with said isolated membrane protein, or said fragment thereof, wherein the isolated membrane protein, or said fragment thereof is solubilized and stabilized by a polymer and/or copolymer, in particular in a polymer and/or copolymer nanodisc;
(b) detecting the formation of a target-protein complex comprising said target and said membrane protein, or said fragment thereof, in the biological sample.

In a second aspect, the present disclosure relates to in vitro diagnostic kits for detecting the presence and/or absence of a target in a biological sample:
(a) at least an isolated membrane protein, or a fragment thereof comprising at least one epitope binding to a target, wherein said isolated membrane protein or said fragment thereof is in the presence of a polymer and/or copolymer that can solubilize and stabilize said membrane protein, in particular the membrane protein is comprised in a polymer and/or copolymer nanodisc; and
(b) reagents for detecting the formation of a target-protein complex between said target and said membrane protein, or said fragment thereof, in said biological sample, wherein said isolated membrane protein or fragment thereof and said reagents are present in an amount sufficient to detect the formation of said target-protein complex.

In a third aspect, the present disclosure relates to solubilized and stabilized isolated membrane proteins, or a fragments thereof, for the use in the treatment of a disease, in particular for the use in the treatment of a disease selected from the group consisting of virus-based diseases, malignant diseases, or chronic inflammatory diseases, such as acute myeloid leukemia, arthritis, COPD including emphysema, intrinsic and extrinsic asthma; cutaneous disease including atopic dermatitis, polymorphic light eruption, SLE; autoimmune diseases, including graft versus host, multiple sclerosis, macrophage activation syndrome, rheumatoid arthritis, juvenile arthritis; intestinal diseases including Crohn's disease and chronic bowel disease, wherein the isolated membrane protein, or said fragment thereof is solubilized and stabilized by a polymer and/or copolymer, in particular in a polymer and/or co-polymer nanodisc.

In a fourth aspect, the present disclosure relates to A test device for the early and rapid detection of a target in a biological sample, wherein the device comprises a test strip, wherein the test strip comprises;
at least one sample application site, in particular a sample pad;
at least one test zone and one control zone, wherein the test zone comprises immobilized isolated membrane proteins, or fragments thereof, wherein the isolated membrane proteins, or said fragments thereof are solubilized and stabilized by a polymer and/or copolymer, in particular in a polymer and/or copolymer nanodisc.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a scheme showing that many pathogenic viruses use the integral membrane proteins of their WNTCP in the membrane of hepatocytes with its natural function as bile acid transporter (left) and used by hepatitis B viruses for binding and internalization via endocytosis as entry port (right)

FIG. 2 is a scheme of a test procedure in which the hepatitis B virus (HBV) binds specifically to the membrane protein NTCP, which is functionally stabilized via a polymer and bound via linkers to, for example, a rapid test membrane.

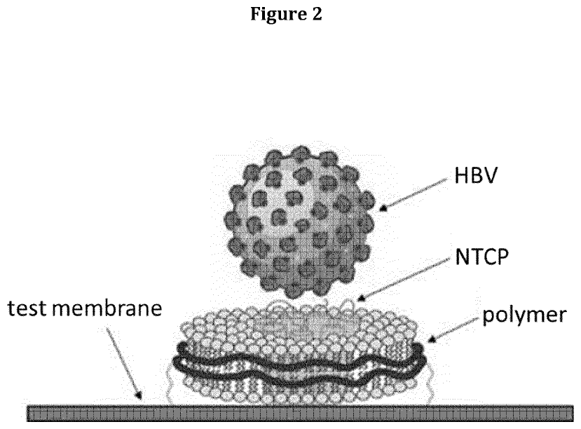

FIG. 3 shows two designs as embodiments of a test device for the rapid detection of a target according to the present disclosure.

FIG. 4 is a scheme of a typical Surface plasmon resonance (SPR) assay, which is an optical-based, label-free detection technology for real-time monitoring of binding interactions between two or more molecules.

FIG. 5 is a scheme showing the kinetic profile of an analyte-target binding reaction.

FIG. 6 is a graph showing the kinetic data and formula for a SPR assay.

FIG. 7 is a graph showing the measurements with Covid in LMNG and ACE2.

FIG. 8 is a graph showing the measurements with Covid in DIBMA and ACE2.

FIG. 9 is a graph showing the measurements with Covid in SMA and ACE2.

FIG. 10 shows a test stripe for a standard lateral flow

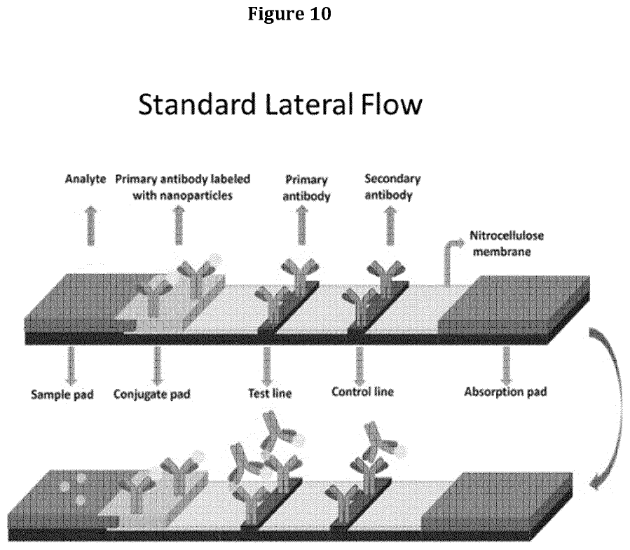

FIG. 11 shows A) a test device according to the present disclosure, wherein in contrast to the standard device of FIG. 10 the primary antibody is changed against stabilized membrane proteins and B) a test device according to the present disclosure, wherein in contrast to the standard device of FIG. 10 the primary antibody labeled with nanoparticles is changed against stabilized membrane proteins.

### DETAILED DESCRIPTION OF THE DISCLOSURE

The present disclosure pertains to in vitro diagnostic methods for detecting the presence and/or absence of a target in a biological sample, wherein said target binds to at least one epitope of an isolated membrane protein, or at least to a fragment of said isolated membrane protein comprising at least one epitope of said isolated membrane protein binding to said target, wherein the method comprises:
(a) contacting said biological sample with said isolated membrane protein, or said fragment thereof, wherein the isolated membrane protein, or said fragment thereof is solubilized and stabilized by a polymer and/or copolymer, in particular in a polymer and/or copolymer nanodisc;
(b) detecting the formation of a target-protein complex comprising said target and said membrane protein, or said fragment thereof, in the biological sample.

For example, virus-receptor interactions play a key regulatory role in viral host range, tissue tropism, and viral pathogenesis. Viruses utilize elegant strategies to attach to one or multiple receptors, over-come the plasma membrane barrier, enter, and access the necessary host cell machinery. The viral attachment protein can be viewed as the “key” that unlocks host cells by interacting with the “lock”—the receptor-on the cell surface, and these lock-and-key interactions are critical for viruses to successfully invade host cells. Many common themes have emerged in virus-receptor utilization within and across virus families demonstrating that viruses often target particular classes of molecules in order to mediate these events. Common viral receptors include sialylated glycans, cell adhesion molecules such as immunoglobulin superfamily members and integrins, and phosphatidylserine receptors. The redundancy in receptor usage suggests that viruses target particular receptors or “common locks” to take advantage of their cellular function and also suggests evolutionary conservation.

Virus-receptor are mostly integral membrane proteins, which are a specific class of proteins, are inserted in vivo into biological membranes and cross the lipid bilayer thereof. The surface of these proteins which naturally comes into contact with the membranes (transmembrane region) is particularly hydrophobic.

Manipulation of membrane proteins in aqueous solution is usually a prerequisite which is essential to their purification and to their structural and functional study. It requires avoidance of the spontaneous aggregation of the hydrophobic domains and maintenance of a relatively non-polar environment around the transmembrane regions. The standard preparations of such proteins in the water-soluble state contain micellar concentrations of surfactants. The success of the process is based on the high affinity of the transmembrane protein regions for these amphiphilic and dispersing compounds. Nevertheless, this is a manipulation which is more intricate than that in the case of soluble proteins, specifically on account of the presence of stabilizing Polymers.

These stabilizing Polymers must be added at a concentration above their critical micelle concentration (cmc) to all the solutions containing the test protein. In addition to any problems of cost posed by the consumption of surfactants, the experiments are often made difficult due to the fact that the membrane proteins are usually fragile and sensitive to their environment. For example, in the presence of an excess of micelles, they can become denatured, while a surfactant defect generally leads to their precipitation.

Several patents, among which mention may be made of WO-A-9,400,557; WO-A-9,115,505; EP-A-363 106; DE-A-3 527 139; JP-A-6,107,6500; U.S. Pat. No. 5,223,411; JP-A-0,227,0856 and JP-A-0,116,8653 describe the extraction, purification and manipulation of membrane proteins in aqueous medium. These proteins are either dispersed in micellar systems or are inserted into lipid bilayers.

Schafmeister et al. Science, 262, pp. 734-738, 1993 have also described the formation of complexes between membrane proteins and amphiphilic peptide polymers. The amphiphilic polymers concerned are small polypeptides known as peptitergents, which have rigid structures (α-helices), one face of which is hydrophobic and the other face hydrophilic. Peptitergents maintain the solubility of bacteriorhodopsin. However, they are unsuccessful in the case of a porin, no doubt because their rigidity limits their possibilities of adaptation when faced with various hydrophobic surfaces. The authors envisage the use of peptitergents to facilitate the crystallization of membrane proteins.

Mention will also be made, in the field of combinations between amphiphilic synthetic polymers and globular (water-soluble) proteins, of the studies by F. Petit et al. Sci., 273, pp. 777-781, 1995 on modified amphiphilic polyacrylates with a molecular weight of between 150,000 and 200,000. The aim of these investigations relates to the study of protein/polymer combinations (formation of gels, kinetics and energetics of the complexation, in particular) rather than to the maintenance of membrane proteins as disperse solutions.

As mentioned above, virus host cell receptors are water-insoluble membrane proteins. To isolate them from the biomembrane and bring them into solution, state-of-the-art solubilizing detergents are used (e.g. SDS, non-ionic glucosides or maltosides, Triton X-100, CHAPS). With these detergents, the lipid bilayer surrounding the membrane protein is destroyed. However, without the surrounding lipid bilayer of the biomembrane, unfolding of the protein often occurs, especially in human membrane proteins, resulting in irreversible destruction of function. Specificity for binding of the virus is also lost.

Surprisingly, it was found that using a polymer and/or a copolymer that can solubilize and stabilize a membrane protein and/or a GPCR in combination with an isolated membrane protein, in particular without any detergent, may be used for in vitro diagnostic methods for detecting the presence of a target in a biological sample.

As mentioned above, the present disclosure pertains also to an in vitro diagnostic kit for detecting the presence and/or absence of a target in a biological sample:
(a) at least an isolated membrane protein, or a fragment thereof comprising at least one epitope binding to a target, wherein said isolated membrane protein or said fragment thereof is in the presence of a polymer and/or copolymer that can solubilize and stabilize said membrane protein, and
(b) reagents for detecting the formation of a target-protein complex between said target and said membrane protein, or said fragment thereof, in said biological sample, wherein said isolated membrane protein or fragment thereof and said reagents are present in an amount sufficient to detect the formation of said target-protein complex.

The terms “polypeptide”, “peptide”, or “protein” are used interchangeably herein to designate a linear series of amino acid residues connected one to the other by peptide bonds between the alpha-amino and carboxyl groups of adjacent residues. The amino acid residues are preferably in the natural “L” isomeric form. However, residues in the “D” isomeric form can be substituted for any L-amino acid residue, as long as the desired functional property is retained by the polypeptide. In addition, the amino acids, in addition to the 20 “standard” amino acids, include modified and unusual amino acids.

In an advantageous embodiment, the membrane proteins according to the present disclosure, in particular the water insoluble membrane proteins are isolated. The term “isolated” when used in relation to a nucleic acid or protein refers to a nucleic acid sequence or protein that is identified and separated from at least one contaminant (nucleic acid or protein, respectively) with which it is ordinarily associated in its natural source.

The polymer used in this process can be a polymer, homopolymer or a copolymer, as will be specified in more detail in the following.

For example, it was found that using a non-aromatic, detergent-free copolymer (DIBMA=diisobutyl-ene/maleic acid copolymer) which, in combination with phospholipids, maintains the native lipid bilayer in which the membrane protein is normally incorporated.

Therefore, membrane proteins can be functionally and detergent-free solubilized and stabilized by wrapping them with a polymer in a so-called nanodisc. Therefore, with DIBMA or DIBMA variants, it should be possible to stabilize host cell viral receptors in nanodiscs in such a way that the specificity for binding the virus is maintained and use as a capture in a rapid test becomes possible.

The term “nanodisc” is well known in the art, and is distinct from the nanodisc clathrates described herein. Nanodiscs are discoidal lipid bilayers encompassed by a protein scaffold. Certain exemplary protein scaffolds are derived from the carboxy-terminal tail of apolipoprotein A-I and is an amphipathic, alpha-helical protein punctuated by prolines (Bayburt, et al., 2004). Mixture of the lipid-free scaffold protein with lipids results in a self-assembled nanoparticle containing a lipid bilayer roughly 10 nm in diameter with two copies of the scaffold protein wrapped around the perimeter of the disc in an anti-parallel fashion. The hydrophobic face of the scaffold protein serves to sequester the hydrocarbon tails of the phospholipids away from solvent (Borhani, et al., 1997). The resulting particle is aqueously soluble and stable. A nanodisc according to the present disclosure may be a polymer-based lipid nanodisc (see e.g. US20190154698A1).

Detergents (like SDS, n-octyl-β-d-glucopyranoside (OG), n-dodecyl-β-d-maltoside (DDM) are widely used in membrane protein solubilization even though it is well known that different detergents have different weaknesses.

Short chain nonionic detergents for example can affect the functional properties of a membrane protein. It seems clear that removing the native lipid bilayer from the membrane protein can interfere with the function of the protein. One way to mimic the native lipid membrane are MSP-nanodiscs or detergent-free polymer systems (Styrene-maleic acid co-polymers (SMAs) (2), Diisobutylene-maleic acid (DIBMA)) (Oluwole, Abraham Olusegun, et al. “Solubilization of Membrane Proteins into Functional Lipid-Bilayer Nanodiscs Using a Diisobutylene/Maleic Acid Copolymer.” Angewandte Chemie International Edition 56.7 (2017): 1919-1924.; Oluwole, Abraham Olusegun, et al. “Formation of lipid-bilayer nanodiscs by diisobutylene/maleic acid (DIBMA) copolymer.” 33.50 (2017): 14378-14388). With the latter you can directly extract membrane proteins from cells without an intermediate step of detergent solubilization. Synthetic polymers have to carry a styrene or maleic acid group himself to solubilize proteins.

As mentioned above, interactions with viral receptors are usually mediated by specific viral attachment proteins expressed on the surface of the virion (Marsh M., Helenius A. Virus entry: open sesame. Cell. 2006; 124:729-740). The inherent differences in the shape (icosahedral or helical) and the com-position of the viral coats (enveloped or nonenveloped) influence the overall architecture of viral attachment proteins. Attachment proteins of enveloped viruses are generally spike-like and extend from the surface of the virion allowing the attachment protein to serve as the first point of contact with the receptor on the plasma membrane. Nonenveloped viruses can either be spherical in nature without extensions, such as polyomaviruses, or be decorated with viral proteins that extend from the virion surface, such as reoviruses. It seems rather apparent that the spike-like protein would be the first contact point between the virus and host cell, in comparison to a viral capsid protein that is embedded on the surface of a spherical viral capsid. However, although reovirus has a spike-like protein that engages cellular receptors, there are additional receptor interactions mediated by capsid components. In addition, while the overall shape of particles can influence attachment protein architecture and the mechanisms by which virions engage cellular receptors, virus-receptor interactions have also been successfully modeled by pseudo-coating viral particles with glycoproteins from an unrelated virus. Pseudotyping viral particles has proved to be a powerful tool for functional analysis of virus-receptor interactions, tissue tropism, and immunity especially for human immunodeficiency viruses (HIV) and highly pathogenic viruses such as Ebola virus (EBOV). Effectively pseudotyping virions that recapitulate patterns of infectious native virions suggests that attachment protein architecture and stoichiometry of attachment protein-receptors is not always essential for viruses to locate and activate the appropriate receptors for infection.

The present disclosure pertains further to an in vitro diagnostic method for detecting the presence of a target virus or a fragment thereof in a biological sample, wherein said virus or said virus fragment comprises a viral attachment protein that binds to at least one epitope of a water insoluble host cell membrane protein, or at least to a fragment of said membrane protein comprising at least one epitope of the membrane protein binding to said viral attachment protein, wherein the method comprises:
(a) contacting said biological sample with the isolated membrane protein, or said fragment thereof, wherein said isolated membrane protein or said fragment thereof is comprised in a DIBMA/lipid particle or in particular in a styrene/maleic acid copolymer;
(b) detecting the formation of a virus-protein complex comprising said target virus or said fragment thereof and said membrane protein, or said fragment thereof, in the biological sample.

Stabilization of a membrane protein is understood as the transfer from a membrane environment to an aqueous solution. Preferably, the lipid environment of the membrane protein is not affected. The structure, binding properties and function are essentially retained. An example could be found in Anaïs Marconnet, Baptiste Michon, Christel Le Bon, Fabrice Giusti, Christophe Tribet, et al., Solubilization and stabilization of membrane proteins by cycloalkane-modified amphiphilic polymers. Biomacromolecules, American Chemical Society, 2020, 21, pp. 3459-3467. ff10.1021/acs.biomac.0c00929ff. ffhal-03018338.

The phrase “stabilized membrane protein” refers to a treated membrane protein so that the protein thermostability improves, or so that the protein retains activity (e.g., of a particular receptor), or maintains a native confirmation, for example, when extracted from a membrane. Stabilizing a membrane protein with an amphiphile as described herein can be, for example, improving its T50 value by about 5° C., about 10° C., about 15° C., about 20° C., or about 25° C., for example, compared to a standard detergent such as DDM. Increasing the stability of an isolated protein is important to allow researchers sufficient time to examine and characterize the protein.

In the following, a general protocol for purification of a membrane protein stabilized in copolymer (e.g. AASTY (Copolymers from styrene and acrylic acid), Ultrasolute Amphipol (polyacrylic acid, partially coupled to amide functions by cycloalkyl amines or cycloalkyl alkylamines) exemplified:

As explained above, the solubilisation, stabilization and purification of membrane proteins out of the native membrane surrounding is dependent on a number of parameters. Most parameters can be optimized during the purification process to a higher efficiency. The parameters include buffer conditions (for example salt, pH), choice of polymer, protein-to-solubilisation agent-ratio, temperature, and time. First, cell lysis and centrifugation is carried out by for example using the following parameters: Adding of protease inhibitors (PI) to buffer and readjust pH value then disrupting cells (e.g., Sonification, French Press). centrifugation at 9 000 rcf for 30 min at 4° C., discarding pellet (cell debris), collecting supernatant, centrifugation of the supernatant at 100 000 rcf for 1 h at 4° C., discarding supernatant and homogenize pellet. Then the solubilisation of membrane proteins is carried out: Polymers form synthetic nanodisc around the protein, thereby maintaining the native phospholipid environment and preserving the native and thus functional properties of the protein in a convenient one step manner (solubilization and stabilization). Detergents on the other hand form micelles around the hydrophobic belt, thus remove the lipids from the surrounding. For native condition the unique lipid environment needs to be conserved.

In one embodiment, the membrane protein is selected from the group consisting of membrane receptor proteins, membrane enzymes, cell adhesion proteins, and transporter proteins, such as ABC transporters, ion channel proteins, water channel proteins (aquaporins), membrane-based ATPases, SLC transporters. That is, as a starting material for the method according to the present invention, a solution of the free polymer is used which stems from the solubilisation, stabilisation and purification of the above-mentioned membrane proteins out of their native surrounding by employing a polymer.

In some advantageous embodiments, the membrane protein is a full-length membrane protein, in particular a water insoluble membrane protein.

In some embodiments, the target to be detected with a method/kit according to the present disclosure comprises moieties which are affinity moieties from affinity substances or affinity substances in their entirety selected from the group consisting of antibodies, antibody fragments, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and their derivatives, mutants or combinations thereof.

In some advantageous embodiments, the method/kit of the present disclosure is used for the diagnosis of a disease selected from the group consisting of virus-based diseases like Coronavirus disease 2019, malignant diseases, chronic inflammatory diseases, such as acute myeloid leukemia, arthritis,

COPD including emphysema, intrinsic and extrinsic asthma; cutaneous disease including atopic dermatitis, polymorphic light eruption, SLE; autoimmune diseases, including graft versus host, multiple sclerosis, macrophage activation syndrome, rheumatoid arthritis, juvenile arthritis; intestinal diseases including Crohn's disease and chronic bowel disease.

Therefore, the target to be detected may be a target virus or a fragment thereof in a biological sample, and wherein said virus or said virus fragment comprises a viral attachment protein that binds to at least one epitope of said membrane protein, or at least to a fragment of said membrane protein. In particular, the virus to be detected is a SARS virus, in particular a SARS-COV-2 or a variant thereof.

In some advantageous embodiments, the method/kit of the present disclosure is used for the diagnosis of a Coronavirus disease like Coronavirus disease 2019 (COVID-19). In some further advantageous embodiments, the biological sample is derived from a human or animal like blood, urine, tissues, organs, saliva, hair, nail clippings, or any other cells or fluids comprising samples.

As pointed out above, the polymer can be a homo polymer or a copolymer. In one embodiment, the polymer can have hydrophilic groups, such as COOH, maleimide, OH, amines, ammonium salts, zwitterions like phosphocholines, and hydrophobic groups, such as polymerized styrene groups, polymerized diisobutylene groups, or linear C1 to C16 (like methyl and ethyl) aliphatic groups, branched C1 to C16 (like isopropyl or t-butyl) aliphatic groups and cyclic C5 to C12 aliphatic or aromatic groups.

The molecular weight of the polymer employed according to the method of the present invention can be 1900 to 20000, for example 2000 to 18000, or 2000 to 15000, or 4000 to 16000, or 4000 to 13000 or 5000 to 14000. The molecular weight can be measured by gel permeation chromatography or mass spectrometry.

Examples for the polymers can be, but are not limited to styrene/maleic acid copolymers, sold by the trade name “SMA”, derivatives of styrene/maleic acid copolymers like SMA 200 and 300, styrene/maleimide copolymers, like SMA 502. These substances can also be functionalized on the COOH groups, with amines, like ethanol amine or ethylene diamine to amides, or with alcohols like glycerol to esters. The polymers can also be functionalized with polyethylene glycols to esters and with aminated polyethylene glycols to amides.

The polymer can be diisobutylidene/maleic acid copolymers, for example DIBMA 10 and DIBMA 12 from Cube Biotech, derivatives of diisobutylidene/maleic acid copolymers, like DIBMA Gly (diisobutylidene/maleic acid copolymers,partially modified with 1-amino-glycerol), DIBMA Glu (diisobutylidene/maleic acid copolymers, partially modified with glucosamine), Glyco DIBMA (diisobutylidene/maleic acid copolymers, partially modified with N-methyl-D-glucamine), and diisobutylidene/maleimide copolymers. DIBMA copolymers can be functionalized with the same molecules like SMA.

Further polymers can be copolymers from styrene and acrylic acid, in particular with a molecular weight of 5.500 and 11.000 and a relation acrylic acid/styrene of 45%/55% to 55%/45%, sold under the name “AASTY”.

Modified polymers from polyacrylic acid can be used, where 10-90% of the carboxylic acid groups can be modified to amides with cyclooctylamine, 2-cyclohexyl-ethylamine, and the like. These substances are sold under the name “Amphipol Ultrasolve”.

In addition to the above disclosure of the polymer, in the following a further description of the polymer is given.

Polymers with Hydrophilic and Hydrophobic Functional Groups:

Examples for hydrophilic groups could be, but are not limited, to polymers of acrylic acid and meth-acrylic acid, maleic acid, carboxylic acid groups in general, amides with α,ω alkylene diamine, ω-hydroxyalkyl amine and ω-aminoalkylthiols, trimethylammonio-alkylamin, amide from carboxylic acid groups with amino-glycerol, TRIS, or Bis-Tris, amide with maltosamine, glucosamine, mannosamine and other amino-functionalized carbo hydrates, or taurine.

Also, esters of carboxylic acid groups with polyethylene glycols, diols, triols, polyols, and carbohydrates can be mentioned.

Other examples can be maleimides, with the nitrogen atom functionalized with alkyl chains with alcohol, thiol, amine, ammonium salts and the like.

Alternatively, zwitterionic molecules, consisting of ammonium and phosphate groups, ca be linked onto carboxylic groups, like it is described in US2020281855A1 or US2021171673A1.

Examples for hydrophobic groups could be, but are not limited, to polymerized styrene and derivatives, such as methylstyrene, diisobutylene and linear and branched alkenes, like 2-propyl, hexyl, octyl, or decyl, coupled to carboxylic groups via ester or amide functions. Also, maleimide groups with alkyl or aryl groups on the amino function are suited examples.

An example for the synthesis of styrene-maleic acid copolymers can be seen in Shintaro Sugai, Nobumichi Ohno, Conformational transitions of the hydrophobic polyacids, Biophysical Chemistry, Volume 11, Issues 3-4, June 1980, Pages 387-395

The use of SMA for building a complex with lipids is described in WO 2006/129127 and references therein. SMA can be purchased at Orbiscope or Cube Biotech, as SMALP 140, SMALP 200, or SMALP 300.

The synthesis of copolymers from diisobutylene and maleic acid anhydride is described in U.S. Pat. No. 4,250,289 by BASF. Hydrolysis of anhydride copolymer to diisobutylene-co-maleic acid is described in Lee, Nature Protocols Vol. 11, No. 7, 2016, 1149-1162, which is described for SMA copolymer, but can be applied to DIBMA without problem.

The synthesis of a DIBMA polymer with a functionalization of a glucosamine on 50% of all carboxy groups can be found on: Bartholomäus Danielczak, Marie Rasche, Julia Lenz, Eugenio Pérez Patallo, Sophie Weyrauch, Florian Mahler, Michael Tope Agbadaola, Annette Meister, Jonathan Oyebamiji Ba-balola, Carolyn Vargas, Cenek Kolar and Sandro Keller, A bioinspired glycopolymer for capturing membrane proteins in native-like lipid-bilayer nanodiscs, DOI: 10.1039/D1NR03811G (Paper) Nanoscale, 2022, 14, 1855-1867. DIBMA can be purchased at Cube Biotech as DIBMA 10 and DIBMA 12.

The preparation of poly(acrylic acid-co-styrene) copolymers is described in WO 2020 257637 and Simon Harrisson *, Francesca Ercole and Benjamin W. Muir, Living spontaneous gradient copolymers of acrylic acid and styrene: one-pot synthesis of pH-responsive amphiphiles, Polym. Chem., 2010, 1, 326-332.

Sometimes the copolymer is a copolymer from styrene and acrylic acid, or a copolymer from styrene and an acrylic acid derivative. Any copolymer derivative may find use in the subject copolymers. Examples for derivatives are acrylates, methacrylates, acrylic esters, acrylamides, and N-substituted acrylamides. In certain cases, the acrylic esters or acrylamides are substituted with a zwitterionic species, as described in U.S. patent application No. 20190062469A1, the disclosure of which is incorporated herein by reference.

In certain embodiments the copolymer contains acrylic acid or an acrylic acid derivative content of from 30% to 70%, 35 to 65%, or 40 to 60%.

The synthesis of Amphipol Ultrasolute, a polyacrylic acid polymer, partially modified by cycloalkyl amines or cycloalkyl-alkylamines, is described in WO 115083 and in Marconnet, A., Michon, B., Le Bon, C., Giusti, F., Tribet, C., & Zoonens, M. (2020). Solubilization and stabilization of membrane proteins by cycloalkane-modified amphiphilic polymers. Biomacromolecules. doi: 10.1021/acs.biomac.0c00929.

Additional polyacrylates, modified with alkyl groups like pentyl, hexyl, and tert-butyl, and their use in forming a complex with a membrane protein are described in US 2020/0383918.

Polymethacrylate, containing butyl Methacrylate (BMA) in Copolymer: ~ 0.52 and methyl acryoloxy choline (MAC) in Copolymer: ~ 0.48, with a degree of polymerization (DP): ~ 39.00, is distributed by Avanti Polar Lipids, with the brand name Polymethacrylate Copolymer (N-C4-52-6.9). Other polymethacrylates are described in Yasuhara K, Arakida J, Ravula T, Ramadugu SK, Sahoo B, Kikuchi JI, Ramamoorthy A. 2017. Spontaneous Lipid Nanodisc Fomation by Amphiphilic Polymethacrylate Copolymers. J Am Chem Soc. 139 (51): 18657-18663.

Polyacrylate polymers, modified with alkanes, such as n-butyl, t-butyl, pentyl, neopentyl, and hexyl are described in Nathaniel Z. Hardin, Thirupathi Ravula, Giacomo Di Mauro, Ayyalusamy Ramamoorthy, Hydrophobic Functionalization of Polyacrylic Acid as a Versatile Platform for the Development of Polymer Lipid Nanodiscs, Small. 2019 March; 15 (9): e1804813. doi: 10.1002/smll.201804813, and US2020383918A1.

Alternatively, linear carbo hydrates with a polymerization degree of less than 100, functionalized with hydrophobic groups, are mentioned in US2022 093587A. Examples for linear carbohydrates are inulin, and examples for hydrophobic groups are alkyl, alkenyl, alkynyl, cycloalkyl, or heteroalkyl having 1-3 hetero atoms. The hydrophobic group is bound to the carbo hydrate via an ether, ester, or amide group.

The present disclosure pertains further to a solubilized and stabilized isolated membrane protein, or a fragment thereof, for the use in the treatment of a disease, in particular for the use in the treatment of a disease selected from the group consisting of virus-based diseases, malignant diseases, or chronic inflammatory diseases, such as acute myeloid leukemia, arthritis, COPD including emphysema, intrinsic and extrinsic asthma; cutaneous disease including atopic dermatitis, polymorphic light eruption, SLE; autoimmune diseases, including graft versus host, multiple sclerosis, macrophage activation syndrome, rheumatoid arthritis, juvenile arthritis; intestinal diseases including Crohn's disease and chronic bowel disease, wherein the isolated membrane protein, or said fragment thereof is solubilized and stabilized by a polymer and/or copolymer, in particular in a polymer and/or copolymer nanodisc.

The interaction of the copolymer stabilized membrane protein and its interaction partner can be detected inclusive but not exclusively via different analytical methods.

Examples for optical detections contain SPR (surface plasmon resonance), RM (resonant mirror), GCI (Grating-Coupled Interferometry), ELISA (enzyme-linked immunosorbent assay) as Direct ELISA, Sandwich ELISA, Competitive ELISA, or Reverse ELISA, and LFA (lateral flow assay).

Surface plasmon resonance (SPR) is the resonant oscillation of conduction electrons at the interface between negative and positive permittivity material in a particle stimulated by incident light. SPR is the basis of many standard tools for measuring adsorption of material onto planar metal (typically gold or silver) surfaces or onto the surface of metal nanoparticles. It is the fundamental principle behind many color-based biosensor applications and lab-on-a-chip sensors.

A good overview for this technology, as well for RM, Dual Polarization Interferometry and other methods is given in “Hikmat N. Daghestani and Billy W. Day; Theory and Applications of Surface Plasmon Resonance, Resonant Mirror, Resonant Waveguide Grating, and Dual Polarization Interferometry Biosensors; Sensors 2010, 10, 9630-9646; doi: 10.3390/s101109630

An ELISA assay uses at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “sandwich” ELISA). After the antigen is immobilized, the detection antibody is added, forming a complex with the antigen. The detection antibody can be co-valently linked to an enzyme or can itself be detected by a secondary antibody that is linked to an enzyme through bioconjugation. Between each step, the plate is typically washed with a mild detergent solution to remove any proteins or antibodies that are non-specifically bound. After the final wash step, the plate is developed by adding an enzymatic substrate to produce a visible signal, which indicates the quantity of antigen in the sample (Wikipedia, English language).

The principle of a lateral flow assay will be described below. Here binding is indicated by a visual signal, which can be produced by almost any dye, but mostly from gold nanoparticles or fluorescent or magnetically labeled particles.

Alternatively, the binding can be detected with a calorimetric method, such as ITC (isothermal titration calorimetry). Isothermal titration calorimetry (ITC) is an analytical technique, a titrimetric method for analysing intermolecular interactions by calorimetric measure. The titration is performed at constant pressure and temperature, meaning that a single ITC experiment offers data on the binding enthalpy, the equilibrium association constant and the stoichiometry, from which the entropy of binding and Gibbs energy can be computed. Hence, a single ITC experiment offers direct access to the key thermodynamic potentials related to the interaction process-Gibbs energy, enthalpy and entropy.

### Test Device

As mentioned above, the present disclosure pertains to a test device for the early and rapid detection of a target in a biological sample, wherein the device comprises a test strip, wherein the test strip comprises;
at least one sample application site, in particular a sample pad.
at least one test zone and one control zone, wherein the test zone comprises immobilized isolated membrane proteins, or fragments thereof, wherein the isolated membrane proteins, or said fragments thereof are solubilized and stabilized by a polymer and/or copolymer, in particular in a polymer and/or copolymer nanodisc.

Therefore, the present disclosure provides a simple and rapid diagnostic test system that improves and simplifies diagnostic assays known in the art. The test device according to the disclosure may comprise a housing which comprises a test strip, wherein the test strip comprises at least one sample application site (e.g. sample pad), at least one test zone (test line) and one control zone (control line).

The housing may comprise openings and/or transparent materials. The openings and/or transparent materials of the housing preferably serve for receiving test samples and for reading the results from the test zone and control zone.

A preferred transparent material of the housing will allow for reading the results from the test zone and control of the test strip. The transparent materials of the housing can also have enclosures which can be opened at the time point of reading. The housing can also completely be made of transparent material. Preferred materials for the test strips are known in the art, such as nitrocellulose membrane, absorbent cellulose pads, blood filter or wicks. The test strip can further comprise a backing layer, such as a polyvinyl backing layer. The materials of the test strips, such as the nitrocellulose membrane and/or absorbent pads can be assembled onto and connected to the backing layer by the means of an adhesive, preferably a pressure sensitive adhesive. In a preferred embodiment the sample application site is a sample pad or a sample wick.

The test strip of the device according to the disclosure may comprise a test zone comprising target antibodies (primary antibodies). The antibodies are preferably immobilized in the respective zone.

There is a preferred device according to the disclosure, wherein the test strip further comprises between the sample application site and the test zones a zone comprising protein-conjugates. The protein-conjugates can be monoclonal or polyclonal antibodies binding to the target. The protein-conjugates in zone will preferably be released when liquid, such as the biological sample, flows through the zone. In a preferred embodiment the protein-conjugates in the zone comprising protein-conjugates comprise gold protein-conjugates. A device is preferred, wherein said comprises gold protein-conjugates.

Furthermore, it is preferred that the test strip comprise said zone comprising protein-conjugates, wherein the protein-conjugates comprise the respective gold protein-conjugates, i.e. anti-target antibody gold conjugates and/or immobilized isolated membrane protein-gold conjugates binding to the target, wherein the isolated membrane proteins, or said fragments thereof are solubilized and stabilized by a polymer and/or copolymer, in particular in a polymer and/or copolymer nanodisc.

In a preferred embodiment the zone comprising protein-conjugates is a fiber glass gold releasing pad, which releases the gold protein-conjugates when liquid, e.g. the sample, flows through it.

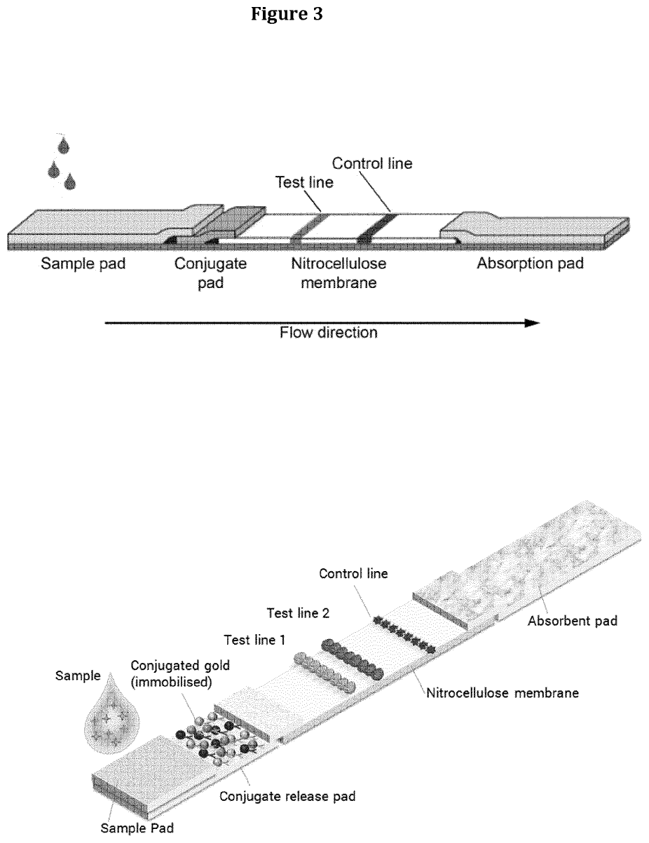

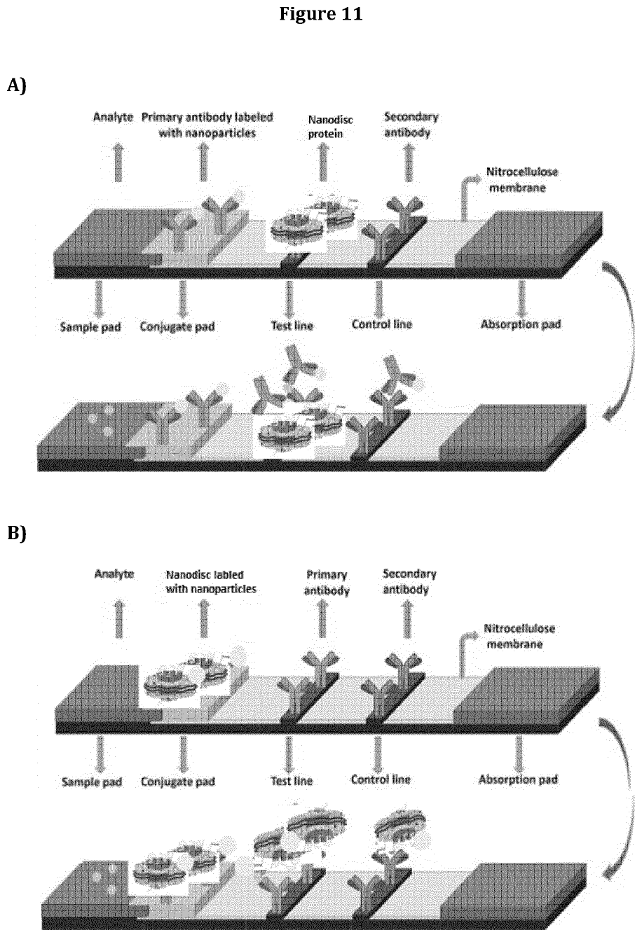

It is further preferred that the control zone of each of the two test strips comprise non-specific capturing antibodies, which are preferably immobilized. Preferred non-specific capturing antibodies are antibodies that capture the conjugated protein non-specifically, anti-mouse antibodies in case mouse c lone conjugates were u sed, anti-rabbit antibodies i n case rabbit clones were used. Other preferred capturing antibodies are anti-(anti-human immunoglobulin) control antibodies. Furthermore, in case of using mouse anti-human immunoglobulin colloidal gold conjugate the control antibody will be anti-mouse immunoglobulin. Preferred embodiments of the test strips of the device of the invention are shown in FIGS. 3 and 11.

A preferred test strip comprises:
a backing layer,
a sample pad as sample application site,
a gold conjugate pad comprising gold protein-conjugates,
a capture line as test zone (test line), wherein the test zone comprises immobilized isolated membrane proteins, or fragments thereof binding to the target, wherein the isolated membrane proteins, or said fragments thereof are solubilized and stabilized by a polymer and/or copolymer, in particular in a polymer and/or copolymer nanodisc,
a control line as control zone,
the material of the test strip in the areas of the test and control zone is a nitrocellulose membrane,
the remaining material of the test strip is an absorbing pad and a blood filter, and
a pressure sensitive adhesive to assemble/connect the sample pad, nitrocellulose membrane, blood filter and absorbing pad to the backing layer.

Another preferred first test strip comprises
a backing layer,
a sample pad as sample application site,
the zone (conjugate pad) comprising protein-conjugates comprises primary antibodies and/or immobilized isolated membrane proteins, or fragments thereof binding to the target, wherein the isolated membrane proteins, or said fragments thereof are solubilized and stabilized by a polymer and/or copolymer, in particular in a polymer and/or copolymer nanodisc,
a test zone (test line) comprising immobilized isolated membrane proteins, or fragments thereof, wherein the isolated membrane proteins, or said fragments thereof are solubilized and stabilized by a polymer and/or copolymer, in particular in a polymer and/or copolymer nanodisc or primary antibodies.
a control line with immobilized non-specific secondary antibody as control zone,
the material of the test strip in the areas of the test and control zone is a nitrocellulose membrane,
the remaining material of the test strip is an absorbing pad and a blood filter, and
a pressure sensitive adhesive to assemble/connect the sample pad, nitrocellulose membrane and absorbing pad to the backing layer.

In a preferred embodiment the gold conjugates in the zone (Conjugate pad) are modified with a com-pound selected from the group comprising chitosan, oligochitosan, glucosamine, polylysine or other polymers or mixtures thereof.

In an advantageous embodiment the test device is defined that a) the conjugate pad comprises primary antibodies against the target labeled with nanoparticles and the test zone comprises immobilized isolated membrane proteins, or fragments thereof binding to the target, wherein the isolated membrane proteins, or said fragments thereof are solubilized and stabilized by a polymer and/or copolymer, in particular in a polymer and/or copolymer nanodisc, or

b) the conjugate pad comprises immobilized isolated membrane proteins, or fragments thereof binding to the target, wherein the isolated membrane proteins, or said fragments thereof are solubilized and stabilized by a polymer and/or copolymer, in particular in a polymer and/or copolymer nanodisc and the test zone comprises primary antibodies against the target.

The action of any of these compounds or mixtures thereof is on the color intensity of the colloidal gold. They are added during the preparation of colloidal gold, but before the conjugation of colloidal gold with protein, i.e. antibody or antigen. The colloidal gold is conjugated after the modification with chitosan (and/or other modifiers) with a specific antibody and/or an antigen. The chitosan and the other modifiers affect the color intensity of colloidal gold and so increase the ability of the human eye to identify the color, and, thus, enable to detect very low concentrations. Signal amplification lies in the range of up to 10 fold. It is preferred to prepare the colloidal gold by the reduction of 1% aqueous solution of tetrachloroauric acid (HAuC14) using trisodium citrate aqueous solution to produce spheroid gold particles. After colloidal gold preparation, chitosan (or any other modifier or mixture) aqueous solution was added with a suitable volume and concentration to convert the color from purple to violet depending on the volume and concentration of the added modification solution.

Therefore, the present disclosure pertains also to a novel rapid test method and to test devices that allow direct detection of a virus from the first day of infection within 15 minutes. Many pathogenic viruses use the integral membrane proteins of their host cells as receptors for attachment to the cell and subsequent uptake and replication in the cell. The part of the membrane protein on the surface of the host cell in question, thus “abused” in its function as a viral receptor, thus determines the ability of a virus to infect and replicate in certain cell types. While the virus itself is highly variable, binding to the host cell receptor represents a highly conserved and unchanging process.

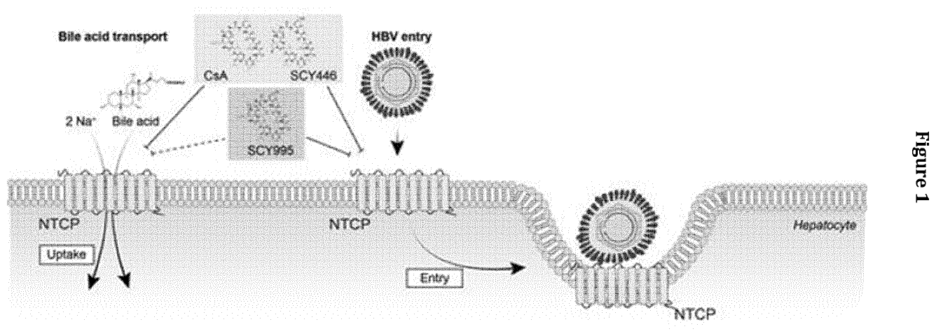

Therefore, stabilized and solubilized host cell membrane proteins are integrated into rapid tests, thus serving as highly specific scavengers for pathogenic viruses and thus building an innovative lateral flow rapid test. The HBV virus, for example, attaches itself to human hepatocytes via the membrane protein NTCP (=sodium taurocholate co-transporting polypeptide or bile acid transporter) anchored in the host cell membrane, in which it multiplies and thus triggers the hepatitis disease (FIG. 1).

In the following, the present invention is further illustrated by referring to the examples and the figures. These examples shall not be construed to limit the invention thereto.

### METHODS AND EXAMPLES

It should be understood that the following examples are for illustrative purpose only and are not to be construed as limiting this disclosure in any manner. All publications, patents, and patent applications cited herein are hereby incorporated by reference in their entirety for all purposes.

### Example 1: Purification of a membrane protein stabilized in copolymer (e.g. poly(acrylic acid-co-styrene) i.e. AASTY, Ultrasolute Amphipol)

The solubilisation, stabilization and purification of membrane proteins out of the native membrane surrounding is dependent on a number of parameters. Most parameters can be optimized during the purification process to a higher efficiency. Parameters include: Buffer conditions (salt, pH etc.), choice polymer, protein-to-solubilisation agent-ratio, temperature, time.

### Cell Lysis and Centrifugation:

Add protease inhibitors (PI) to buffer and readjust pH value then disrupt cells (e.g., Sonification, French Press). Centrifuge at 9 000 rcf for 30 min at 4° C., discard pellet (cell debris), collect supernatant. Centrifuge supernatant at 100 000 rcf for 1 h at 4° C., discard supernatant and homogenize pellet.

### Solubilisation of Membrane Proteins:

Polymers form synthetic nanodiscs around the protein, thereby maintaining the native phospholipid environment and preserving the native and thus functional properties of the protein in a convenient one step manner (solubilization and stabilization). Detergents on the other hand form micelles around the hydrophobic belt, thus remove the lipids from the surrounding. For native condition the unique lipid environment needs to be conserved.

If solubilisation efficiency is low it is advised to screen variation of parameters to improve the yield of total solubilised protein. A standard protocol is described as follows:

Add solubilisation agent to the protein solution
Ideal concentrations may vary, good starting points are:
0.5%-5% SMA/DIBMA
0.1-2.5% UltraSolute Amphipol
0.1-2.5% AASTY
Solubilise for 3 h to 24 h at 4° C. while stirring
Higher temperatures can be screened for optimization
Centrifuge at 100 000 rcf for 1 h at 4° C.
Discard pellet, collect supernatant
Use solubilized membrane protein in polymer nanodisc (supernatant) for affinity chromatography

### Example 2: Affinity Chromatography

Polymers can interfere in the binding of protein to the binding matrix, therefore a reduction of polymer concentration is advantageous. For sufficient binding a polymer concentration of 0.25% is essential. A further decrease down to 0.025%-0.050% polymer may be advantageous for an improved binding efficiency.

Purify protein of choice via preferred protocol depending on affinity tag.

### Example 3: Diagnostic with Copolymer Supported Membrane Proteins

Protocol for standard ELISA assay utilizing proteins solubilized with co-polymers
the co-polymer-stabilized membrane protein(s) of choice need to be in a purified state (multiple days at 4° C.).
the day before the ELISA assay, a sterile 96-well-plate has to be coated with protein (concentration of 500 ng/well to 1 μg/well is a good starting point for optimization). 100 μL of protein solution are pipetted into each well, except for possible controls such as a blank or a coat consisting of 1% BSA in TBS to visualize any possible unspecific interactions.
Additionally, it is recommended to perform double determinations for each condition tested.
The plate is left to rest at least overnight at 4° C., covered with either Parafilm or a plastic lid to avoid evaporation or drying out.
The following day, the washing buffer is prepared by dissolving 0.05% Tween20 in TBS buffer [50 mM Tris, 100 mM NaCl; pH 7.0].
coated plate is washed three times [300 μL per well]: two times with TBS containing Tween20 and once with TBS without the additional detergent. Between changes of the buffer, the plate is left to rest for about 5 minutes each time.
After washing, 300 μL of 3% milk powder in TBS or 3% BSA in TBS are added to each well and incubated for 1 hour at room temperature to block the plate. The choice between milk powder and BSA is dependent on coating protein, ligand as well as antibodies utilized in the assay.
Following this blocking step, the washing steps previously described are repeated in the same manner.
The chosen ligand which can be a protein, an antibody, etc. is diluted to the concentrations of choice. A serial dilution is recommended using the same buffer the protein has been stored in.
100 μL of each dilution of ligand are pipetted into the corresponding wells and incubated for one hour at room temperature.
The washing steps are repeated once again after this incubation.
Following these washing steps, 100 μL of the first antibody targeting the ligand is added to each well and incubated for one hour at room temperature.
The dilution should be chosen based on the manufacturer's suggestion. If this information is not available, a dilution of 1:1000 using 3% milk powder or BSA is recommended.
Incubation of the first antibody can be omitted if a conjugate of two antibodies is being used for the assay.
Once more, the same washing steps are performed and are followed up by incubation of, again, 100 μL of now the second antibody for one hour at room temperature. Here, diluting of the antibody to 1:1000 is always performed by using 3% milk powder in TBS.
After incubation of the second antibody, the plate is washed again. This time, two times with 0.05% Tween20 in TBS buffer, two times with simple TBS and a last washing step with MilliQ water [300 μL per well in each washing step].
As the visualization/development buffer has to be as fresh as possible, it is recommended to mix the ingredients for the solution right after the last washing step with water. For the visualization/development buffer 9 mL of MilliQ water, 1 mL of 1M Sodium Acetate [pH 6.0, adjusted by using 1M citric acid], 62.5 μL 3,3′,5,5′-Tetramethylbenzidine [abbr.: TMB, 10 mg/mL in DMSO] and 15 μL 3.5% H₂O₂ are mixed and used immediately by pipetting 100 μL of the solution into each well.
Incubation of the visualization/development buffer is flexible but should be at least 10 minutes yet not exceed 90 minutes. However, the shorter the incubation time, the more visible differences between the different samples will be.

I The enzymatic reaction is stopped by adding 100 μL of 10% sulfuric acid. Mixing of the solutions inside the wells can be increased by lightly tapping against the frame of the plate. After five minutes, results can be obtained by measuring the plate at 450 nm with a standard photometer.

### Example 4: Protocol for SPR Assay Utilizing Membrane Proteins Solubilized with CoPolyMers (COV Spike Protein)

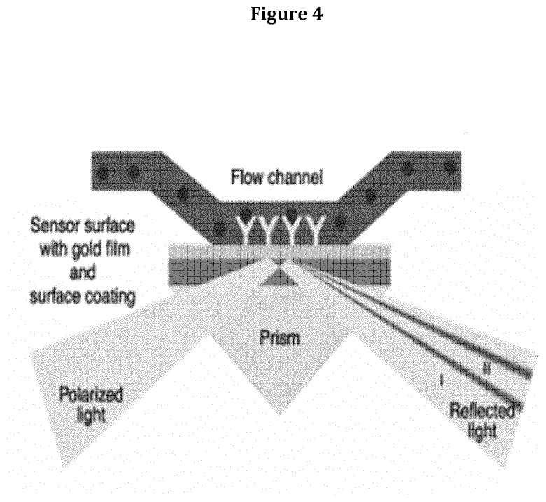

Surface plasmon resonance (SPR) is the resonant oscillation of conduction electrons at the interface between negative and positive permittivity material in a particle stimulated by incident light (FIG. 4). SPR is the basis of many standard tools for measuring adsorption of material onto planar metal (typically gold or silver) surfaces or onto the surface of metal nanoparticles. It is the fundamental principle behind many color-based biosensors applications and lab-on-a-chip sensors. It should be stressed that SPP is not a resonance on the planar surface and it is a polariton or surface-wave like phenomenon.

How the SPR works:
Optical method measuring changes in the mass of biomolecules
Biomolecule of interest is bound to a metal film within a flow chamber

Analytes are directed through the flow chamber
Upon binding an analyte, the refractive index of the metal film changes
   changed reflection angle of light
“the surface plasmon resonance phenomenon”
The more is bound, the greater the angle offset
   The greater the measured signal

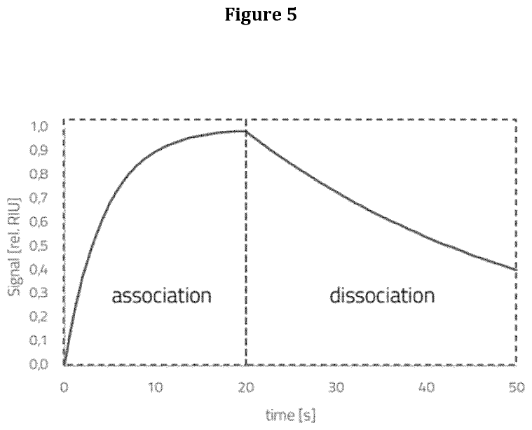

How is SPR used?
Protein is associated to one channel of a gold plate
E.g. EDC/NHS coupling
Ligand/Protein partner is prepared in increasing concentrations
Ligands are then directed over both channels to allow for an association
Association is detected by an increasing signal
After a defined time, buffer without ligand is directed as well and the dissociation is measured
Disassociation is detected by a decreasing signal (FIG. 5).

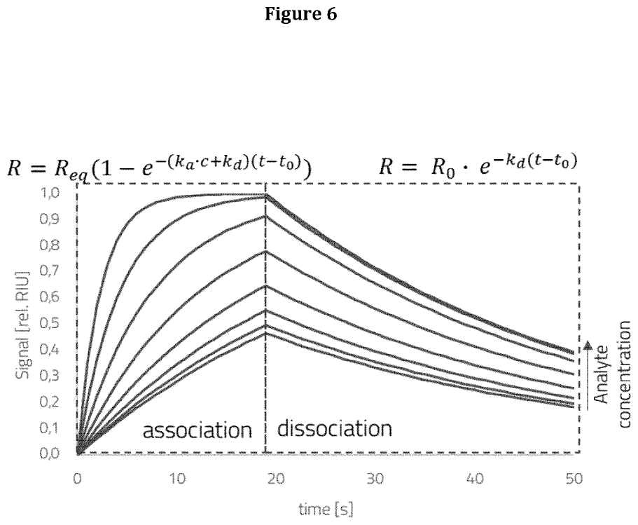

How to determine kd- and ka-value?
The signal is mainly dependent on:
   association rate (ka)
   dissociation rate (kd)
   amount of bound ligand
   concentration of analyte
With a known ligand concentration and multiple different analyte concentration ka and kd are able to be calculated (FIG. 6).
Example for fit equations *:

$$R = {R}_{eq} ( 1 - {e}^{- ( {k}_{a} \cdot c + {k}_{d} ) ( t - {t}_{0} )} )$$

$$R = {R}_{0} \cdot {e}^{- {k}_{d} ( t - {t}_{0} )}$$

Simplified integrated equations for a reversible 1:1 binding of ligand and analyte

### Example 5: Measurements with COVID and ACE

ACE receptor ectodomain is fused to chip-Sars COV2 Spike Protein stabilized in either LMNG, DIBMA or SMA is washed over ACE fused chip. Association and Dissociation capacity is measured in different concentrations:
SPR Biosensor SR7500DC (Reichert)
Chip: Indigo Chip (Cube Biotech GmbH https://cube-biotech.com/), prepared by chemical modification of a dextran chip (SCR 200R-DCM5, Xantec GmbH, Düsseldorf, Germany) according the procedure from WO2020/109162, example 7.
Wash chip with ACE2 buffer until baseline is stable
Immobilize ACE2 ectodomain on left channel of chip (approx. 600 RU)
60 g/mL, flow rate: 0.5 ml/min
Wash chip with ACE2 buffer until baseline is stable
Exchange buffer to COVID buffer and wash both channels until baseline is stable
Analyte: COVID spike protein in DIBMA
Prepare COVID sample concentrations: 0 nM; 0,488 nM; 0.96 nM; 1.9 nM; 3.9 nM; 7.8 nM; 15.6 nM; 31.25 nM; 72.5 nM 125 nM; 250 nm; 500 nM;
Start measurement:
25 L/min.
Association time: 6 min
Dissociation time: 11 min.
experiment referenced twice: empty channel without ACE2 and injection of running buffer (buffer blank)

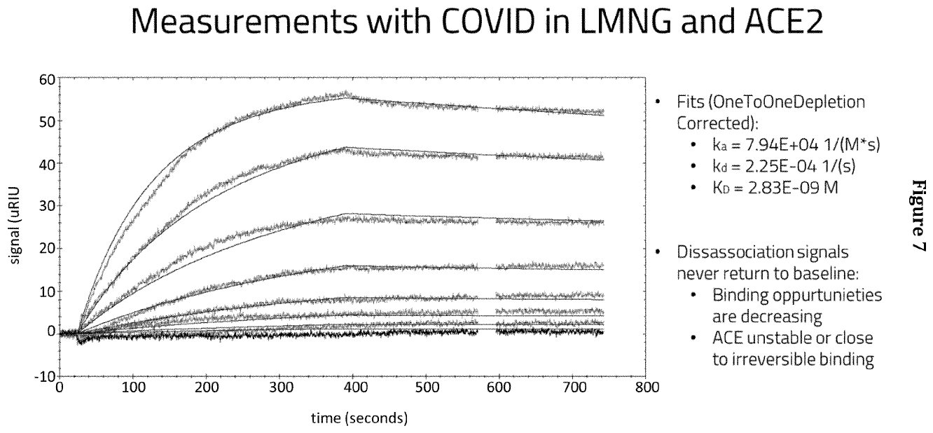

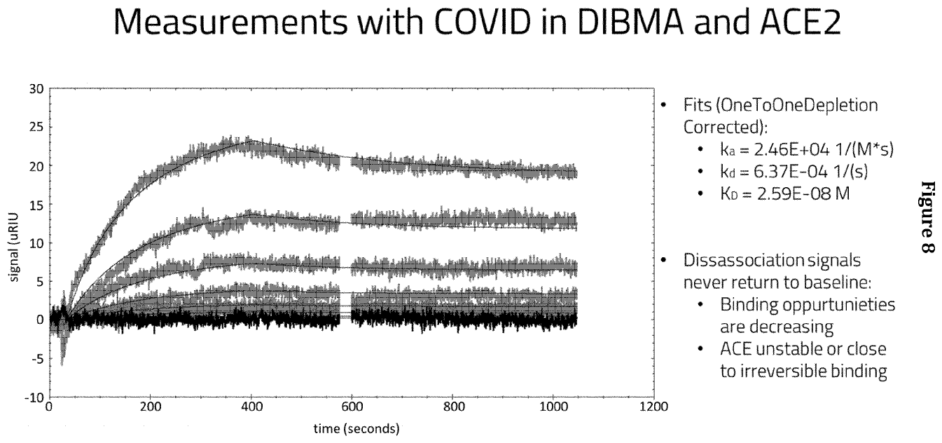

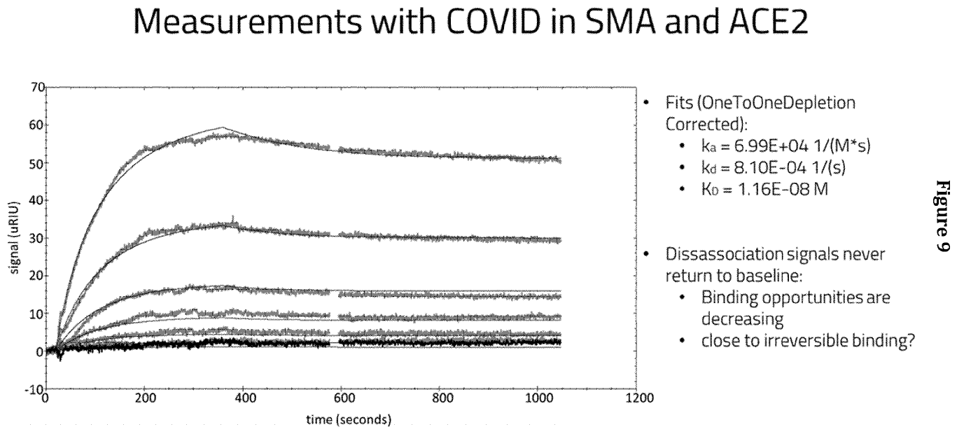

The results of the measurements are shown in FIGS. 7, 8 and 9.

### Overview and Comparison

The results in Table 1 show:
COVID in LMNG showed weaker dissociation, than samples solubilized in polymer (lowest kd-value)

COVID in LMNG and in SMA show comparable association, therefore COVID in SMA is active
COVID in DIMBA showed weakest association (lowest ka-value)
COVID in SMA showed strongest dissociation (highest kd-value)

TABLE 1

| | COVID IN | COVID in | COVID in |
|---|---|---|---|
| | LMNG | DIBMA | SMA |
| kₐ [1/(M*s)] | 7.94E+04 | 2.46E+04 | 6.99E+04 |
| k_{d} [1/s] | 2.25E−04 | 6.37E−04 | 8.10E−04 |
| K_{D} [M] | 2.83E−09 | 2.59E−08 | 1.16E−08 |

The experiments show that COVID in SMA successfully stabilizes the Spike Protein in a conformation where it can bind the ACE receptor on the chip.

### Advantage of Membrane Proteins in Polymer/Copolymer/Nanodisc:

If SPR is performed with 2 full length membrane proteins both proteins cannot be stabilized in detergent since a wash with detergent buffer might lead to permanent dissociation of coated Protein A from the chip. Copolymers stabilizes the membrane proteins in their native lipid environment without the need of adding copolymer to every buffer.

1. An in vitro diagnostic method for detecting the presence and/or absence of a target in a biological sample, wherein said target binds to at least one epitope of an isolated membrane protein, or at least to a fragment of said isolated membrane protein comprising at least one epitope of said isolated membrane protein binding to said target, wherein the method comprises:
(a) contacting said biological sample with said isolated membrane protein, or said fragment thereof, wherein the isolated membrane protein, or said fragment thereof is solubilized and stabilized by a polymer and/or copolymer, in particular in a polymer and/or copolymer nanodisc; and
(b) detecting the formation of a target-protein complex comprising said target and said membrane protein, or said fragment thereof, in the biological sample.

2. The method according to claim 1, wherein the membrane protein is selected from the group consisting of membrane receptor proteins like G protein-coupled receptors (GPCRs), membrane enzymes, cell adhesion proteins, and transporter proteins, such as ABC transporters, ion channel proteins, water channel proteins (aquaporins), membrane-based ATPases and SLC transporters.

3. The method according to claim 1, wherein the membrane protein is a full-length membrane protein.

4. The method according to claim 1, wherein the membrane protein is a water-insoluble membrane protein.

5. The method according to claim 1, wherein said polymer/copolymer that solubilize and stabilize said membrane protein has hydrophilic groups, such as COOH, maleimide, OH, amines, ammonium salts, zwitter ions like phosphocholines, and hydrophobic groups, such as polymerized styrene groups, polymerized diisobutylene groups, or linear (methyl, ethyl, up to C16) branched (isopropyl, t-butyl) and cyclic (C5 to C12) aliphatic or aromatic groups.

6. The method according to claim 1, wherein the molecular weight of the polymer/copolymer is 1900 to 20000.

7. The method according to claim 1, wherein the polymer/copolymer is selected from the group consisting of diisobutylene/maleic acid copolymer, styrene maleic acid copolymer, (acrylic acid-co-styrene) copolymer and polyacrylic acid, partly coupled to cyclic alkylamines or cycloalkylalkylamines, in particular a styrene/maleic acid copolymer.

8. The method according to claim 1, wherein the membrane protein or said fragment thereof is comprised in a polymer/lipid particle like a DIBMA/lipid particle.

9. The method of claim 8, wherein the DIBMA in the DIBMA/lipid particle is glucosamine- and/or aminoglycerol-modified DIBMA.

10. (canceled)

11. The method according to claim 1, wherein the method is used for the diagnosis of a disease selected from the group consisting of virus-based diseases like Coronavirus disease 2019, malignant diseases, chronic inflammatory diseases, such as acute myeloid leukemia, arthritis, COPD including emphysema, intrinsic and extrinsic asthma; cutaneous disease including atopic dermatitis, polymorphic light eruption, SLE; autoimmune diseases, including graft versus host, multiple sclerosis, macrophage activation syndrome, rheumatoid arthritis, juvenile arthritis; intestinal diseases including Crohn's disease and chronic bowel disease.

12. The method according to claim wherein the target is a target virus or a fragment thereof in a biological sample, and wherein said virus or said virus fragment comprises a viral attachment protein that binds to at least one epitope of said membrane protein, or at least to a fragment of said membrane protein.

13. The method of claim 12, wherein said virus is a SARS virus, in particular a SARS-COV-2 or a variant thereof.

14. The method according to claim 1, wherein the method is used for the diagnosis of a Coronavirus disease like Coronavirus disease 2019 (COVID-19).

15. The method according to claim 1, wherein the biological sample is derived from a human or animal like blood, urine, tissues, organs, saliva, hair, nail clippings, or any other cells or fluids comprising samples.

16. An in vitro diagnostic kit for detecting the presence and/or absence of a target in a biological sample:
(a) at least an isolated membrane protein, or a fragment thereof comprising at least one epitope binding to a target, wherein said isolated membrane protein or said fragment thereof is in the presence of a polymer and/or copolymer that can solubilize and stabilize said membrane protein, in particular the membrane protein is comprised in a polymer and/or copolymer nanodisc; and
(b) reagents for detecting the formation of a target-protein complex between said target and said membrane protein, or said fragment thereof, in said biological sample, wherein said isolated membrane protein or fragment thereof and said reagents are present in an amount sufficient to detect the formation of said target-protein complex.

17. (canceled)

18. (canceled)

19. (canceled)

20. The kit of according to claim 16, wherein said polymer/copolymer that can solubilize and stabilize said membrane protein and/or a GPCR has hydrophilic groups, such as COOH, maleimide, OH, amines, ammonium salts, zwitter ions like phosphocholines, and hydrophobic groups, such as polymerized styrene groups, polymerized di isobutylene groups, or linear (methyl, ethyl, up to C16) branched (isopropyl, t-butyl) and cyclic (C5 to C12) aliphatic or aromatic groups.

21. (canceled)

22. (canceled)

23. The kit according to claim 16 wherein the membrane protein or said fragment thereof is comprised in a polymer/lipid particle like a DIBMA/lipid particle.

24-44. (canceled)

45. A test device for the early and rapid detection of a target in a biological sample, wherein the device comprises a test strip, wherein the test strip comprises;
at least one sample application site, in particular a sample pad;
at least one test zone and one control zone, and
at least a zone as a conjugate pad comprising gold protein-conjugates,
wherein in the test zone and/or the conjugate pad comprises immobilized isolated membrane proteins, or fragments thereof, wherein the isolated membrane proteins, or said fragments thereof are solubilized and stabilized by a polymer and/or copolymer, in particular in a polymer and/or copolymer nanodisc.

46. (canceled)

47. The test device according to claim 45, wherein
a) the conjugate pad comprises primary antibodies against the target labeled with nanoparticles and the test zone comprises immobilized isolated membrane proteins, or fragments thereof binding to the target, wherein the isolated membrane proteins, or said fragments thereof are solubilized and stabilized by a polymer and/or copolymer, in particular in a polymer and/or copolymer nanodisc, or
b) the conjugate pad comprises immobilized isolated membrane proteins, or fragments thereof binding to the target, wherein the isolated membrane proteins, or said fragments thereof are solubilized and stabilized by a polymer and/or copolymer, in particular in a polymer and/or copolymer nanodisc and the test zone comprises primary antibodies against the target.

48. The test device according to claim 45, wherein the test strip comprises between the sample application site and the test zone, a zone comprising gold protein-conjugates, the protein in the gold protein-conjugates are antibodies against the target and/or immobilized isolated membrane proteins, or fragments thereof binding to the target, wherein the isolated membrane proteins, or said fragments thereof are solubilized and stabilized by a polymer and/or copolymer, in particular in a polymer and/or copolymer nanodisc.

49-66. (canceled)
